(19) United States
(12) Patent Application Publication
(10) Pub. No.: US 2018/0113137 A1
(43) Pub. Date: Apr. 26, 2018

(54) METHOD FOR DETECTING THE QUANTITY OF BIOMARKER AND IDENTIFYING DISEASE STATUS

(71) Applicant: TCM BIOTECH INTERNATIONAL CORP., New Taipei City (TW)

(72) Inventors: PEI-JER CHEN, Taipei City (TW); SHIOU-HWEI YEH, Taipei City (TW); CHIAO-LING LI, Taipei (TW); DING-SHINN CHEN, Taipei City (TW); YA-CHUN WANG, New Taipei City (TW); SHENG-TAI TZENG, New Taipei City (TW)

(21) Appl. No.: 15/821,864

(22) Filed: Nov. 24, 2017

Related U.S. Application Data
(63) Continuation-in-part of application No. 14/515,550, filed on Oct. 16, 2014.
(60) Provisional application No. 61/892,796, filed on Oct. 18, 2013.

(51) Int. Cl. G01N 33/574; C12Q 1/6886; C12Q 1/6806; C12Q 1/6809; C12Q 1/686; G01N 33/576

(52) U.S. Cl. CPC G01N 33/57488; C12Q 2600/112; C12Q 1/6806; C12Q 1/6809; C12Q 1/686; G01N 33/5761; C12Q 2600/16; C12Q 2600/158; G01N 2800/56; G01N 2800/7028; C12Q 2600/106; G01N 2800/085; G01N 2800/52; C12Q 2600/118; C12Q 1/6886

(57) ABSTRACT

The present invention provides a chimera nucleic acid obtained from circulatory system for monitoring tumor size. The nucleic acid comprises partial sequence derived from host genome and partial sequence derived from non-host genome. The partial sequence derived from host genome and the partial sequence derived from non-host genome form a chimera junction. The chimera junction is obtained from cell-free nucleic acids and is indicative of tumor size.

### CROSS REFERENCE TO RELATED APPLICATION

This application is a continuation-in-part of U.S. application Ser. No. 14/515,550, filed Oct. 16, 2014, which claims priority to U.S. Provisional Application No. 61/892,796, filed Oct. 18, 2013, and the contents of which are incorporated by reference herein.

### REFERENCE TO AN ELECTRONIC SEQUENCE LISTING

The contents of the electronic sequence listing (US62260_ST25.txt; Size: 13.6 KB; and Date of Creation: Nov. 22, 2017) is herein incorporated by reference in its entirety.

### FIELD

The present disclosure relates generally to the field of using circulating cell-free nucleic acids in a subject to identify and monitor a disease development in the subject.

### BACKGROUND

The fundamental cause of a tumor or a cancer has been attributed to genetic alterations caused by hereditary or environmental factors. These genetic alterations, once ir-repaired or irreparable, will accumulate and eventually cause normal cells to become malignant cells. As a tumor or a cancer develops its own unique spectrum of genetic alterations; therefore, monitoring of the alterations can provide information about the tumor or the cancer.

Both normal and malignant cells undergo cycles of turnover where chromosomes of dead cells are fragmented and released into body fluids, for example blood circulation, as circulating cell-free nucleic acids. Sequencing of the chromosome fragments indicates that the circulating cell-free nucleic acids from the blood or serum of patients carry the genetic alterations from the original tumor or cancer.

The conventional design of using host genome sequences containing specific genetic alterations as indicators for capturing tumor/cancer-specific nucleic acid sequences from total circulating cell-free nucleic acids works for an advanced tumor/cancer, where the tumor/cancer is sufficiently lame and a significant amount of tumor/cancer-specific nucleic acid sequences (more than 5% of total circulating nucleic acids) is released into the circulation. Given its limited amount (0.01% to 1% in total blood), the circulating cell-free nucleic acids is hard to detect even in the advanced tumor/cancer. As a result, for early or intermediate stage of the tumor/cancer, the proportion of the tumor/cancer circulating cell free nucleic acids is too low to be reliably detected. Moreover, tumor/cancer-specific mutations are usually single-base mutations, small insertions or deletions which are very difficult to be separated from nucleic acid sequences without such mutations released from the non-tumor/cancer somatic cells. In other words, not all circulating cell free nucleic acids bear the altered genetic information; most of the circulating cell free nucleic acids is unaltered and from host genome.

Conventional approach for cancer/'tumor nucleic acid detection sampled from genomic DNA. Murakami et al. presented an approach to detect HBV DNA in liver and peripheral blood mononuclear cells (Murakami, Y., Minami, M., Daimon, Y., & Okanoue, T. (2004). Hepatitis B virus DNA in liver, serum, and peripheral blood mononuclear cells after the clearance of serum hepatitis B virus surface antigen. Journal of medical virology 72(2), 203-214.). In this approach, Alu-PCR were used to detect covalently closed circular HBV DNA, HBV core DNA, HBV S DNA, and HBV X DNA in genome DNA extracted from peripheral blood mononuclear cells and liver tissue. The product of Alu-PCR was sequenced to determine the presence of above targets. In this approach, known HBV DNA regions from patient's genome were amplified by PCR and sequenced.

For the ex-vivo detection of HBV DNA integration, Lin et al. presented an approach to detect HBV DNA integration sites in liver cancer cell lines (Lin, S., Jain, S., Block, T., Song, F., & Su, Y. H. (2013). Detection of clonally expanded HBV DNA integration sites as a marker for early detection of HBV related HCC.). Lin et al. disclosed a target enrichment assay and cloned DNA constructs to identify known HBV DNA integration sites in Hep3B cell line. The HBV DNA containing nucleotide position 1571 to 1960 were captured by biotinylated HBV RNA baits and cloned, and each of the clones were sequenced. In this approach, known HBV DNA integrations sites in Hep3B cell line were detected from cloning and sequencing.

### BRIEF DESCRIPTION OF THE DRAWINGS

Implementations of the present technology will now be described, by way of example only, with reference to the attached figures. The aspect of the present disclosure can be better understood with reference to the following figures. The components in the figures are not necessarily drawn to scale with the emphasis instead being placed upon clearly illustrating the principles of the present disclosure.

FIG. 1 schematically shows general progression of virus-infected cells.

FIG. 2 schematically shows an exemplary method of obtaining target circulating cell-free nucleic acids.

FIG. 3 illustrates the specificity of viral-host junction.

FIG. 4 shows the changes in the amount of a specific viral-host junction before and after tumor resection.

FIG. 5 illustrates the specificity of viral-host junction.

FIG. 6 and FIG. 7 show the concentration of specific viral-host junction in a subject before tumor resection.

FIG. 8 and FIG. 9 show the concentration of specific viral junction in another subject before tumor resection.

FIG. 10, FIG. 11, FIG. 12 and FIG. 13 show the concentration of specific viral junction in another subject before tumor resection.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising,” when utilized, means “including, but not necessarily limited to”; it specifically indicates open-ended inclusion or membership in the so-described combination, group, series and the like.

The term “subject” refers to an object of studies or experimental samples, may include human, monkey, groundhog or any animal. The term “integration” or “integrant” is making up or being a part of a whole. The integrant herein means single nucleic acid base pair, a part of nucleic acid sequence, a fragment of nucleic acid sequence or a gene sequence from a viral chromosome embed in a host chromosome sequence. The term “junction” is a combination of a fragment of a viral chromosome sequence and a part of a host chromosome sequence. The term “read number” refers to the times of next generation sequencing (NGS) reads. Each NGS read is analyzed to see whether it contained junction sequences or not. The reads containing junction sequences are then assembled into the finalized junction sequences. Therefore, each finalized junction sequence is assembled by the NGS reads containing the same junction region, and the accumulated read number represents the number of reads assembled into that specific finalized junction sequence. The term “amount” means the relative quantification of copy numbers of the specific junction cfDNA fragment in subject serum or plasma. The specific junction sequences are obtained from NGS analyzed results. According to the sequence, the specific junction cfDNA fragment from serum or plasma are detected by droplet digital PCR (ddPCR) platform in absolute quantification which can estimate the concentration (copy numbers) of the specific junction cfDNA fragment in each milliliter serum or plasma.

The present disclosure is described in relation to the innovation of using circulating cell-free nucleic acids in a subject to identify and monitor a disease development in the subject.

Certain human tumors/cancers, for example hepatocellular carcinoma (HCC), are caused by chronic infection of hepatitis B virus (HBV). These tumors/cancers accumulate genetic alterations in their genomes. Among such alterations, a unique one is the integration of viral genome into the host genome, usually occurring in the early stage of infections. Superimposed upon these mutations are other somatic mutations that continue to occur and finally transform the cells to tumors/cancers.

As noted, when HCC cells turn over, fragmented genetic nucleic acids will be released into the body fluids, comprising blood, urine or interstitial fluid. Circulating cell free nucleic acids which floats freely in the circulatory system, for example blood circulation, usually comprises DNA fragments. These fragments include those from host genome, from viral genome, and/or from the viral integration sites, for example the viral-host junction.

Infected cells, for example HBV-infected hepatocytes, proliferate if they become cancerous and so is the amount of the viral integrants carried by the infected cells. The amount of viral integrants thus is in proportion to the size of tumor/cancer in general. In addition, as the viral integrates into the host genome at different sites, each tumor/cancer carries a unique spectrum of viral integration sites. The viral integration sites and/or the viral-host junction, are cancer/tumor-specific and can be used to identify and monitor the tumor/cancer development.

In one embodiment, human subjects are employed in the tests to illustrate the present invention. Subject 1 has a 12×10×9 (cm) tumor diagnosed by computer tomography. According to the histological report when Subject 1 is employed in this test, Subject 1 is defined as a Grade III HCC patient. Subject 2 has a 18×13.5×9 (cm) tumor diagnosed by computer tomography. According to the histological report, Subject 2 is defined as a Grade III HCC patient. Subject 3 has a 8×7.5×7 (cm) tumor identified by computed tomography. According to the histological report, Subject 3 is defined as a Grade III HCC patient. Subject 4 has a 2×2×2 (cm) tumor and is at Grade II. Subject 5 has a tumor smaller than 2×2×2 (cm) and the stage of the cancer development is not determined and/or not available at the time of test enrollment. Subject 6 is defined as a Grade III HCC patent, and has a tumor size of 9.1 cm³. Subject 7 is defined as a Grade II HCC patent, and has a tumor size of 11 cm³. Subject 8 has a tumor size of 3 cm³. Subject 9 has a tumor size of 11.58 cm³. Subject 10 has a tumor size of 4.6 cm³.

In another embodiment, multiple blood samples are obtained from human subjects. Each time, blood is drawn, collected in a clinically suitable container and, if needed, stored in a suitable condition for later analysis. Each blood sample is processed to obtain serum, such as by centrifugation. The cfDNA can be extracted by using a commercial kit, comprising MagNA Pure LC Total Nucleic acid Isolation kit (Roche). The tumor tissues are obtained. Genomic DNAs of tumor cells are extracted.

In another embodiment, in order to proportionally amplify the all ctDNA obtained from the human subject, the ctDNA can be attached or ligated with at least one adaptor to at least one end or both ends of the ctDNA. The ligating at least one adaptor to at least one end or both ends of the ctDNA can be performed by using TruSeq DNA Sample Preparation (Illumina), TruSeq Nano DNA LT Library Preparation Kit (Illumina), IonTorrent (Life Technologies) or other equivalent reagents.

In another embodiment, after the ctDNA is ligated with at least one adaptor, each ctDNA in the sample from the human subject can be amplified by using TruSeq DNA Sample Preparation (Illumina), TruSeq Nano DNA LT Library Preparation Kit (Illumina). IonTorrent (Life Technologies) or other equivalent reagents.

In another embodiment, polynucleotides having HBV genome sequence are used as probes here. The probes can be either designed or synthesized from the fragmentation of HBV genome. The probes portfolio can be cover the whole HBV genome sequence. The whole HBV genome sequences can be obtained from the National Center for Biotechnology Information. The probes can be synthesized by using a commercial kit, comprising Ion TargetSeq Custom Enrichment Kit (Life Technologies). The length of the probes is synthesized in a range from about 20 bases to about 200 bases. The length of the probes is preferably synthesized in a range from about 50 bases to about 180 bases. After the synthesis of the probes, each probe can be labeled, comprising biotinylated, at least one end of the probe. Biotinylation of probes can be performed by using a commercial kit, comprising ruSeq Nano DNA LT Library Preparation Kit (Illumina), Ion TargetSeq Custom Enrichment Kit (Life Technologies) or other equivalent kit. The probes can be subsequently attached or linked to a bead, for example through biotin.

In another embodiment, the all amplified ctDNA are mixed and incubated with the beads coated with the biotinylated probes to allow hybridization between the ctDNA and the biotinylated probes. The certain ctDNA that have at least partial viral sequences anneal to the complementary sequences on the probes and can form a bead-probe-ctDNA complex. The other ctDNA that does not bind to the probes float freely and does not form any complex. The bead-probe-ctDNA complexes are separated from non-binding ctDNA by, for example, centrifugation. The bead-probe-ctDNA complexes are obtained. After the bead and the probe are removed from the bead-probe-ctDNA complexes, target ctDNA can be collected. Capturing of the ctDNA hybridized with the probes can be performed by using TargetSeq Hybridization & Wash Buffer Kit (Life Technologies) or other equivalent kit.

In another embodiment, in order to sequence and identify the target ctDNA. The target ctDNA can be further sequenced using lonTorrent platform, HiSeq 2500 (Illumina) or other equivalent sequencing platform.

In another embodiment, the copy number of specific target ctDNA junctions are quantified by BIO-RAD Eva Green Droplet Digital PCR kit. The copy number of each of the specific target ctDNA junction in the subject can be determined. Other equivalent DNA quantification kits can also be used.

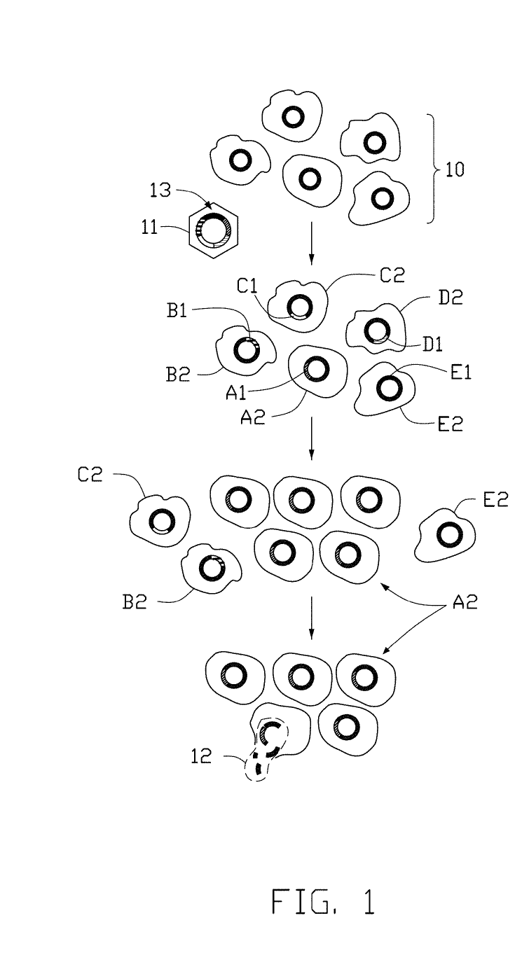

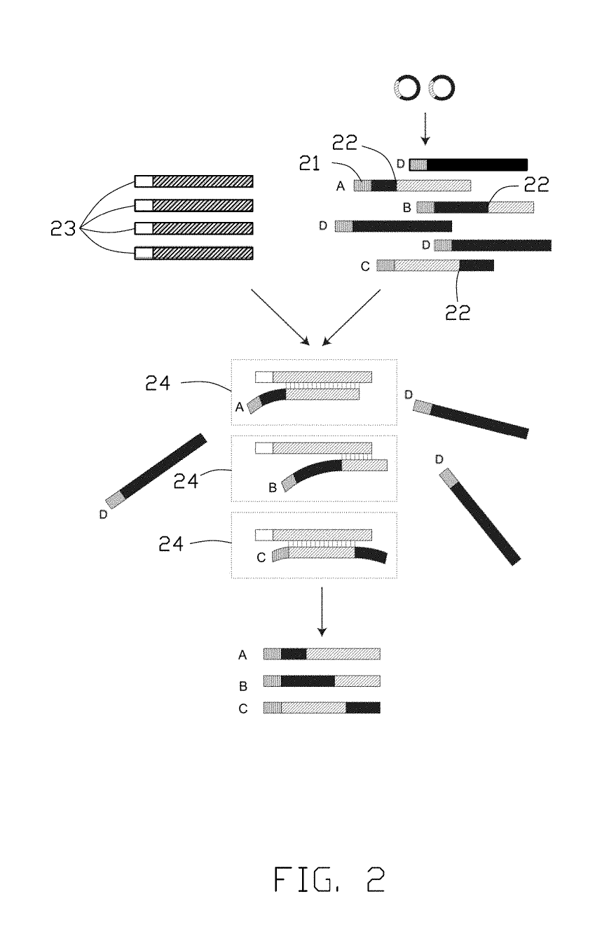

FIG. 1 illustrates a cancer development process of cells. Referring to FIG. 1, hepatocytes 10 in a subject generally have the same host genome. Referring again to FIG. 1, hepatocytes 10 comprise a plurality of hepatocytes A2, B2, C2, D2 and E2. Upon HBV 11 infection, HBV 11 can integrate its viral genome 13 into the host genome of the infected hepatocytes. Parts of HBV genome 13 are integrated into the host genome, generating infected hepatocytes with different viral integration sites and different integrated viral gene sequence. As show in FIG. 1, viral sequence A1 is integrated into cell A2, viral sequence B1 is integrated into cell B2, viral sequence C1 is integrated into cell C2, viral sequence D1 is integrated into cell D2 and viral sequence E1 is integrated into cell E2. The integration of HBV DNA sequences can create viral-host junctions in the host cell genome. Infected cells A2, B2, C2, D2 and E2 grow, develop and accumulate additional genetic alterations with time. Both host and viral sequences, altered or not, might lead to proliferation, stable stage or cell death. Cell A2 carries the alterations that induce malignant transformation and lead to proliferation or clonal expansion. It is to be noted that a viral-host junction can lead to malignant transformation, or may be an insignificant integration that does not lead to proliferation.

Referring again to FIG. 1, the infected cell A2 proliferates, expands in cell number and transforms into a malignant cell, which subsequently forms a cancerous or tumorous cell cluster. Cells B2, C2, D2, E2 do not go through malignant progression and remain in very small population or die out. The all infected cells A2 bear the same hereditary information, comprising the host genome, at least partial viral genome, and the viral-host junctions. If the infected cells A2 proliferate, the number of cell A2-specific viral-host junctions will increase proportionally in general. The same viral-host junctions are present in the same cancerous cell lineage whether they trigger cancer development or not. As depicted schematically in FIG. 1, the cancerous clone goes through rapid proliferation and turnover, and some of the infected cells A2 rupture and die. DNA strands 12 of these ruptured and dead infected cells A2 are released into circulatory system or body fluids, such as blood. These DNA strands 12 become fragmented, float freely through the circulatory system and become a part of circulating cell-free DNA (cfDNA) in blood stream. As used herein, with reference to the present application, it shall be clearly understood that the terms “circulating cell-free DNA”, “circulatory cell-free DNA” and “cfDNA” refer to DNA that is obtained from the blood stream or the circulatory system of a subject or a patient, wherein the DNA that is obtained from the blood stream or the circulatory system of the subject or the patient is either substantially free of other cellular components, or is essentially entirely free of other cellular components. Some of the cfDNA is later on digested or cleaned by functional cells such as macrophages while some remain in the blood stream especially when the cfDNA is in large amount. cfDNA fragments originated from tumor or cancer cell is “circulatory tumor DNA”, “circulating tumor DNA” and “ctDNA”. By examining and/or detecting the ctDNA in the circulatory system or the body fluids, one can obtain information about tumor/cancer development.

FIG. 2 illustrates a schematic view of isolating target ctDNA. Circulating DNA from dead tumor/cancer cells is released into blood in fragments as ctDNA. The cfDNA is collected and ligated with adaptors 21, and forms cfDNA A, B, C and D. The cfDNA is amplified by using any suitable approach, for instance, using a primer complementary to the sequence of the adaptor 21 in an appropriate amount. It is to be noted that preferred amplification methods amplify all cfDNA in a similar or the same proportions so that the amplified cfDNA provides genuine information as to the amount and the ratio of every kind of cfDNA existing in the blood. In FIG. 2, sequences derived from viral genome are designated in hatch area while sequences derived from host genome are designated in black.

The amplified cfDNA can be categorized into cfDNA having only host genome sequences (cfDNA D), cfDNA having only viral genome sequences (not shown), and cfDNA having both viral and host genome sequences and thus comprising viral-host junctions 22 (cfDNA A, B, and C). According to a preferred approach, all amplified cfDNA are incubated with polynucleotide probes 23 (designed and derived from the viral genome sequence) to allow hybridization to occur. It is to be noted that the polynucleotide probes 23 may have different sequences even though all drawn alike. Referring again to FIG. 2, the cfDNA having viral genome sequence alone or the cfDNA having at least one viral-host junction (such as cfDNA A, B and C) can form probe-cfDNA complexes 24. These complexes can be separated from the cfDNA that does not hybridize with the probe (such as cfDNA D). The target ctDNA is the cfDNA having only viral genome sequence or the cfDNA having at least one viral-host junction which are then obtained and separated from the complexes. The sequences of target ctDNA are further obtained. Tissue origins of the target ctDNA are identified based on tissue tropism and specificity of virus infection.

Table 1 shows top ten target sequences identified in the DNA samples obtained from Subject 1 tumor tissue. As shown, a junction sequence is inserted into the host chromosome (Host Chromosome #) at a specific integration position (Integration Position) with an accumulated read number (Accumulated Reads). Accumulated read number is obtained by NGS sequencing result. Sequences having the same junction are counted to give the number of the junction present in the sample. Each sequence includes at least partial viral chromosome sequence (underlined) and a partial host chromosome sequence to form a viral-host junction.

TABLE 1
Junction Data of Subject 1 Tumor Tissue

| | Host | | | |
|---|---|---|---|---|
| | Chromosome | Integration | | Accumulated |
| # | # | Position | Junction Sequence | Reads |
| 1 | 17 | 22247083 | GGTCTTACATAAGAGGACTCAGAAAATACTTT | 290 |
| | | | GTGATGAT | |
| 2 | 17 | 22252195 | AACTCCTTTTGAGAGCGCAGTGTTCGGTGCAG | 234 |
| | | | GTCCCCAG | |
| 3 | 1 | 121360041 | ATCATCACAAAGTATTTTCTGAGTCCTCTTAT | 192 |
| | | | GTAAGACC | |
| 4 | 12 | 118876274 | TGAGGTGAGAGGATCTCTTGAGCACAGATGAT | 115 |
| | | | GGGATAGG | |
| 5 | X | 58568585 | AAACGTCCACTTGCAGATTTTATGTAATTGGA | 102 |
| | | | AGTTGGGG | |
| 6 | 8 | 56895765 | AGCAGGAAAATATATGCCCCACCTTCCCTTTC | 106 |
| | | | TCTGACCC | |
| 7 | 1 | 121475300 | AGGAAGACTGCCTACTCCCACAGGCCTGAAAG | 85 |
| | | | CGCTCCAA | |
| 8 | X | 58563641 | AGCATTCGGGCCAGGGTTCACCTCAGGCTCAG | 67 |
| | | | GGCACATTG | |
| 9 | 16 | 21525068 | GCATTTGGTGGTCTATAAGCACACCCGCCCAC | 38 |
| | | | ACCAATCT | |
| | 18 | 77932557 | CAAGACCAGCCTGAGGATGACCTGTCTCTTAG | 26 |
| 10 | | | AGGTGGAGA | |

Table 2 shows the target sequences identified in the ctDNA samples obtained from the serum of Subject 1. The ctDNA samples are obtained from Subject 1 13 days before a tumor excision. As shown, each sequence contains at least partial viral chromosome sequence (underlined) and a partial host chromosome sequence to form a viral-host junction.

TABLE 2
Junction Data of Subject 1 Serum Sample

| | Host | | | |
|---|---|---|---|---|
| | Chromosome | Integration | | Accumulated |
| # | # | Position | Junction Sequence | Reads |
| 11 | 17 | 22251295 | CACTCCTTTTGAGAGCGCAGTGTTCAGGTG | 94 |
| | | | CAGGGTCCCC | |
| 12 | 1 | 121360041 | ATCATCACAAAGTATTTTCTGAGTCCTCTT | 82 |
| | | | ATGTAAGACC | |
| 13 | 1 | 13727 | AACAGAAAGATTCGTCCCCAAATCCAATCT | 68 |
| | | | GTCTTCCATC | |
| 14 | 8 | 56895765 | AGCAGGAAAATATATGCCCCACCTTCCCTT | 62 |
| | | | TCTCTGCCCT | |
| 15 | 17 | 22247083 | GGTCTTACATAAGAGGACTCAGAAAATACT | 42 |
| | | | TTGTGATGAT | |
| 16 | 16 | 21525068 | GCATTTGGTGGTCTATAAGCACACCCGCCC | 31 |
| | | | ACACCAATCT | |
| 17 | 8 | 56895953 | ATCATCCTGGGCTTTCTGCACTTCCCATAG | 16 |
| | | | GTAATCAAAG | |
| 18 | X | 58563641 | AGCATTCGGGCCAGGGTTCACTCAGGCTCA | 9 |
| | | | GGGCACATTG | |

As illustrated in Tables 1 and 2, at least #3 (from tumor sample) and #12 (from serum sample), #1 (from tumor sample) and #15 (from serum sample), and #2 (from tumor sample) and #11 (from serum sample) each pair have the same viral-host junction sequences. Similar patterns (including the relative read numbers) of viral-host junction sequences identified in both tumor DNA and ctDNA indicate that chimera ctDNA in serum is derived from tumor DNA. By selectively enriching the ctDNAs carrying at least a portion of the viral genome in the serum, viral-host junctions are identified to provide tumor-specific information about the subject.

Table 3 shows the target sequences identified in the DNA samples obtained from Subject 2 tumor tissue. As shown, each sequence contains at least partial viral genome sequence (underlined) and partial host genome sequence and forms a viral-host junction.

TABLE 3
Junction Data of Subject 2 Tumor Tissue

| | Host | | | |
|---|---|---|---|---|
| | Chromosome | Integration | | Accumulated |
| # | # | Position | Junction Sequence | Reads |
| 1 | 3 | 111653312 | ATGAAGCTATTTATAATAAAACAAACTTTATTAAA | |
| | | | TCTAGTTTAAATGCCTTACTCTCTTTTTTGCCTTC | 4183 |
| | | | TGACTTCTTTCCTTCTATTCGAGATCTCCT | |
| 2 | 2 | 80278757 | TTTCATTGTTGCTGTTTTTCAAATTGATTTTGGGA | 3772 |
| | | | TCCAGCCTGTTATTCTACTCCCTTAACTTTCATGG | |
| | | | GATATGTAATTGGAAGTTGGGGTACTTTACC | |
| 3 | 3 | 111653206 | TCTCCCTTTAGACTTCAAACACTTCAAAATATGAC | 1269 |
| | | | TTCACTACAAAGCTTTATAGAATGCCAGCCTTCCA | |
| | | | CAGAGTATGTAAATAATGCCTAGTTTTGAA | |
| 4 | 2 | 80278655 | CCAGCACATTTGTCTATAAATTTACATTCTTGGAT | 752 |
| | | | ATTAGCAAAATTGCAAACAGACCAATTTATGCCTA | |
| | | | CAGCCTCCTAGTACAAAGACCTTTAACCTA | |
| 5 | 1 | 189879551 | TCCAGTGTTTGTGGGTTGAGCAGTATTATTGCATG | 485 |
| | | | GCCCAGTGGTGGTGGTTGATGTTCCTGGAAGTAGA | |
| | | | GGACAAACGGGCAACATACCTTGGTAGTCC | |
| 6 | 1 | 189879474 | TGCAAGTGGTTGCAGTTCTTTTGCTTTGCCACCAC | 174 |
| | | | CACTGGGCCATGCAAAACCTGCACGATTCCTGCTC | |
| | | | AAGGAACCTCTATGTTTCCCTCTTGTTGCT | |
| 7 | 20 | 60227034 | CAGGAGGAGGTGATGGACCCACTGGGTGGTGAAGA | 169 |
| | | | ACAGTTTCTCTTCCAAAATTACTTCCCACCCAGGT | |
| | | | GGCCAGATTCATCAACTCACCCCAACACAG | |
| 8 | 22 | 26941239 | ATCTGTAAAATTGGGATCATCACACTTTCCTTTTA | 100 |
| | | | TTGGGGTTTAAATGAATACCCAAAGACAAAAGAAA | |
| | | | ATTGGTAATAGAGGGTAAAAAGGGACTCAAG | |
| 9 | 20 | 60227112 | TGGCCGAGGCCATCTTCTAAATAAATGTGTGGAAG | 93 |
| | | | AGAAACTGTTCTTCAGTATTTGGTGTCTTTTGGAG | |
| | | | TGTGGATTCGCACTCCTCCCGCTTACAGAC | |
| 10 | 5 | 1295309 | AGGACGGGTGCCCGGGTCCCCAGTCCCTCCGCCAC | 37 |
| | | | GTGGGAAGCGCGGTCCAGACCAATTTATGCCTACA | |
| | | | GCCTCCTAGTACAAAGACCTTTAACCTAAT | |

Table 4 shows the target sequences identified in the ctDNA samples obtained from serum of Subject 2. Serum samples are obtained from Subject 2 at tumor excision. As shown, each sequence contains at least partial viral genome sequence (underlined) and partial host genome sequence and forms a viral-host junction.

TABLE 4
Junction Data of Subject 2 Serum Sample

| | Host | | | |
|---|---|---|---|---|
| | Chromosome | Integration | | Accumulated |
| # | # | Position | Junction Sequence | Reads |
| 11 | 3 | 111653312 | ATGAAGCTATTTATAATAAAACAAACTTTATTAAAT | 3277 |
| | | | CTAGTTTAAATGCCTTACTCTCTTTTTTGCCTTCTG | |
| | | | ACTTCTTTCCTTCTATTCGAGATCTCCT | |
| 12 | 20 | 60227034 | CAGGAGGAGGTGATGGACCCACTGGGTGGTGAAGAA | 642 |
| | | | CAGTTTCTCTTCCAAAATTACTTCCCACCCAGGTGG | |
| | | | CCAGATTCATCAACTCACCCCAACACAG | |
| 13 | 1 | 189879551 | TCCAGTGTTTGTGGGTTGAGCAGTATTATTGCATGG | 373 |
| | | | CCCAGTGGTGGTGGTTGATGTTCCTGGAAGTAGAGG | |
| | | | ACAAACGGGCAACATACCTTGGTAGTCC | |
| 14 | 2 | 50012582 | GTCCGTTGGTGGTGAACTGGGCAAGATAATTGCATG | 372 |
| | | | GCCCAGTGGTGGTGGTTGATGTTCCTGGAAGTAGAG | |
| | | | GACAAACGGGCAACATACCTTGGTAGTC | |
| 15 | 15 | 48344568 | AGATTGGTCTATAATTTTCTTTTACTATCTTCAGTA | 237 |
| | | | TTTGGTATCTTTGGGAGTGTGGATTCGCACTCCTCC | |
| | | | CGCTTACAGACCACCAAATGCCCCTATC | |
| 16 | 2 | 80278757 | TTTCATTGTTGCTGTTTTTCAAATTGATTTTGGGAT | 230 |
| | | | CCAGCCTGTTATTCTACTCCCTTAACTTCATGGGAT | |
| | | | ATGTAATTGGAAGTTGGGGTACTTTACC | |
| 17 | 20 | | TGGCCGAGGCCATCTTCTAAATAAATGTGTGGAAGA | 209 |
| | | | GAAACTGTTCTTCAGTATTTGGTGTCTTTTGGAGTG | |
| | | | TGGATTCGCACTCCTCCCGCTTACAGAC | |
| 18 | 1 | 189879474 | TGCAAGTGGTTGCAGTTCTTTTGCTTTGCCACCACC | 205 |
| | | | ACTGGGCCATGCAAAACCTGCACGATTCCTGCTCAA | |
| | | | GGAACCTCTATGTTTCCCTCTTGTTGCT | |
| 19 | 2 | 50012660 | GTAAGCCATTGTGGCTTTCCTGACCAGCCCACCACC | 205 |
| | | | ACTGGGCCATGCAAAACCTGCACGATTCCTGCTCAA | |
| | | | GGAACCTCTATGTTTCCCTCTTGTTGCT | |
| 20 | 2 | 80278655 | CCAGCACATTTGTCTATAAATTTACATTCTTGGATA | 64 |
| | | | TTAGCAAAATTGCAAACAGACCAATTTATGCCTACA | |
| | | | GCCTCCTAGTACAAAGACCTTTAACCTA | |

As illustrated in Tables 3 and 4, at least #1 (from tumor sample) and #11 (from serum sample), #7 (from tumor sample) and #12 (from serum sample), and #5 (from tumor sample) and #3 (from serum sample) both have the same viral-host junction sequences. Similar patterns of viral-host junction sequences identified in both tumor DNA and ctDNA show that chimera ctDNA in serum is derived from tumor DNA. By selectively enriching the target ctDNA in the serum, viral-host junctions are identified to provide tumor-specific information about the subject.

Table 5 shows the target sequences identified in the DNA samples obtained from Subject 3 tumor tissue. As shown, each sequence contains at least partial viral genome sequence (underlined) and partial host genome sequence and forms a viral-host junction.

TABLE 5
Junction Data of Subject 3 Tumor

| | Host | | | |
|---|---|---|---|---|
| | Chromosome | Integration | | Accumulated |
| # | # | Position | Junction Sequence | Reads |
| 1 | 5 | 1295930 | GGAAATGGAGCCAGGCGCTCCTGCTGGCCGC | 3024 |
| | | | GCACCGGGCGCCTCACACCAGAACATCGCAT | |
| | | | CAGGACTCCTAGGACCCCTGCTCGTGTTACA | |
| | | | GGCGGGG | |
| 2 | 8 | 111636420 | TCAAGCAGAAAAACCATGAAGATTTAAAAAC | 635 |
| | | | TTGTAAATATTTGAATGTGGGCTCCACCCCA | |
| | | | ACAGTCCCCCGTGGGGAGGGGTGAACCCTGG | |
| | | | CCCGAAT | |
| 3 | 14 | 52591737 | CTAAGGGACACTACAGGAAACCAGCCCCGAA | 354 |
| | | | GTGATTTCTTTTGAAATTCCAAATCTTTCTG | |
| | | | TCCCCAATCCCCTGGGATTCTTCCCCGATCA | |
| | | | TCAGTTG | |
| 4 | 9 | 138857330 | CCTCGAAGCCTGTGCCAACCTAGCCCATTCC | 190 |
| | | | TCAGGCTCAGGGCCTCCTCACATCTGTGCCA | |
| | | | GCAGCTCCTCCTCCTGCCTCCACCAATCGGC | |
| | | | AGTCAGG | |
| 5 | 1 | 68549419 | CATTGTTACTGTGATATGCTATAATTATTCT | 188 |
| | | | CACCTTATGTGTCCAAGGAATACTAACATTG | |
| | | | AGATTCCCGAGATTGAGATCTTCTGCGACGC | |
| | | | GGCGATT | |
| 6 | 9 | 31455679 | ATGGAGAATACAGCACATTATTAGGAGTAAG | 172 |
| | | | TTTCCTTAAACACATTTTGATTTTTTGTACA | |
| | | | ATATGTTCCTGTGGCAATGTGCCCCAACTCC | |
| | | | CAATTAC | |
| 7 | 17 | 714434403 | TTTGCCACCTTCCTGCCACTTTGTAGATGCA | 138 |
| | | | AGATCTTGGGCAAGTTCCCGTGGGCGTTCAC | |
| | | | GGTGGTTTCCATGCGACGTGCAGAGGTGAAG | |
| | | | CGAAGTG | |
| 8 | 12 | 126230889 | CAGTGGAAACAAAGCCACTGGGAAGTTCAAA | 135 |
| | | | CTGAGAGAAGCCCACCACAAGTCTAGACTCT | |
| | | | GTGGTATTGTGAGGATTTTTGTCAACAAGAA | |
| | | | AAACCCC | |
| 9 | X | 35911295 | AGTATATCATCAGTTATTTTTCAAGGTTTTC | 124 |
| | | | TAAGTAAACAGTTTCTCAACCTTTACCCCGT | |
| | | | TGCTCGGCAACGGCCTGGTCTGTGCCAAGTG | |
| | | | TTTGCTG | |
| 10 | 10 | 75397400 | TCAGGGAGGGGATGTTGACTGCATTTTGGAG | 58 |
| | | | GTTCAGGGCCTACTAACAACTGTGCCAGCAG | |
| | | | CTCCTCCTCCTGCCCTCCACCAATCGGCAGT | |
| | | | CAGGAAGG | |

Table 6 shows the target sequences identified in the ctDNA samples obtained from serum of Subject 3. Serum samples are obtained from Subject 3 at tumor excision. As shown, each sequence contains at least partial viral genome sequence (underlined) and partial host genome sequence and forms a viral-host junction.

TABLE 6
Junction Data of Subject 3 Serum Sample

| | Host | | | |
|---|---|---|---|---|
| | Chromosome | Integration | | Accumulated |
| # | # | Position | Junction Sequence | Reads |
| 11 | 5 | 1295930 | GGAAATGGAGCCAGGCGCTCCTGCTGGCCGCG | 153 |
| | | | CACCGGGCGCCTCACACCAGAACATCGCATCA | |
| | | | GGACTCCTAGGACCCCTGCTCGTGTTACAGGC | |
| | | | GGGG | |
| 12 | 8 | 111636420 | TCAAGCAGAAAAACCATGAAGATTTAAAAACT | 52 |
| | | | TGTAAATATTTGAATGTGGGCTCCACCCCAAC | |
| | | | AGTCCCCCGTGGGGAGGGGTGAACCCTGGCCC | |
| | | | GAAT | |
| 13 | 21 | 47565536 | CCCGGGACCGACCCCAGGAAGAGCCAGGGGCC | 27 |
| | | | CGGGTGATCCCTGCGGGGGTCTGGCTTTCAGT | |
| | | | TATATGGATGATGTGGTATTGGGGGCCAAGTC | |
| | | | TGTA | |
| 14 | 21 | 28573066 | AATGAAAATCTCATTGATTTTTCACTTATAGG | 25 |
| | | | TTTTACCTTAGAGCTCCTCCTCTGCCTAATCA | |
| | | | TCTCATGTTCATGTCCTACTGTTCAAGCCTCC | |
| | | | AAGC | |
| 15 | 7 | 87842849 | AGAATTGATACCTAAGCTGAGCAGAAATGAGG | 24 |
| | | | CCGACCATGAAGTGAGTGCCTAATCATCTCAT | |
| | | | GTTCATGTCCTACTGTTCAAGCCTCCAAGCTG | |
| | | | TGCC | |
| 16 | 7 | 148503201 | CGTAGGAAAGACAAGGTGGCATTGATGGAAAG | 19 |
| | | | CAGTAGTTTTTGAGCCCTTCGCAGACGAAGGT | |
| | | | CTCAATCGCCGCGTCGCAGAAGATCTCAATCT | |
| | | | CGGG | |
| 17 | 1 | 162277132 | TTAAAAAGGAGTTTTGTTTGTTAGTCTATTCA | 16 |
| | | | CTCATTTCAAGGAACATAGAAGAAGAACTCCC | |
| | | | TCGCCTCGCAGACGAAGGTCTCAATCGCCGCG | |
| | | | TCGC | |
| 18 | 12 | 125048731 | CAGTTCCCTGGCTCCAAGCTCCCTCAAAAGAT | 15 |
| | | | GCCCAGCTGGCCTTTCCCAAAGGCCTTGTAAG | |
| | | | TTGGCGAGAAAGTAAAAGCCTGTTTTGCTTGT | |
| | | | ATAC | |
| 19 | 7 | 30412226 | ACATGCCCTTCACTTCAGCCTGATGCTCCTGG | 13 |
| | | | CATAAGCTCAGCAATTTTGGAGTGCGAATCCA | |
| | | | CACTCCAAAAGACACCAAATATTCAAGAACAG | |
| | | | TTTC | |
| 20 | 13 | 84505952 | AATTTCCCCTGAATAGCTGCAGTACTCACAGA | 13 |
| | | | CACACTGGATGCTACTCACCTCTGCCTAATCA | |
| | | | TCTCATGTTCATGTCCTACTGTTCAAGCCTCC | |
| | | | AAGC | |

As illustrated in Tables 5 and 6, similar patterns of viral-host junction sequences identified in both tumor DNA and ctDNA show that ctDNA in serum is derived from tumor DNA. By selectively enriching the target ctDNA in the serum, viral-host junctions are identified to provide tumor-specific information about the subject.

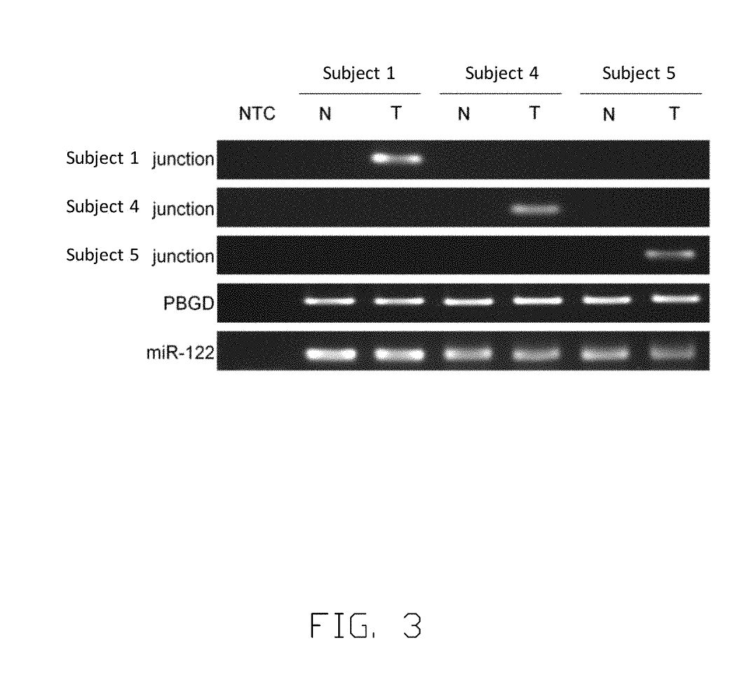

FIG. 3 illustrates that the genomic DNA of Subject 1, Subject 4 and Subject 5 are processed and analyzed by polymerase chain reaction (PCR). The genomic DNA (gDNA) from tumor tissues and non-tumor tissues is obtained. One chimera DNA sequence in tumor gDNA is identified and selected in each subject to serve as a target to conduct the tests. Specifically, the chimera DNA sequence of Subject 1 used in this analysis is

GGTCTTACATAAGAGGACTCAGAAAATACTTTGTGATGAT
(viral genome sequence underlined),

### Subject 4

ACTTCAAAGACTGTGTGTTTCTAATTATTTTGGGGGACAT,

### and Subject 5

GTAGGCATAAATTGGTCTGTACCTCACTTCCCTGCTTTCC.

The presence of the three specific viral-host junctions is determined in the tumor gDNA (T) and non-tumor gDNA (N). Porphobilinogen deaminase (PBGD) and miR-122 are used as internal control. No-template control (NTC) is also included. As illustrated in FIG. 3, the specific viral-host junction of Subject 1 is only present in tumor gDNA (T) but not in non-tumor gDNA (N). Same patterns are observed in Subject 4 and Subject 5, indicating that the identified viral-host junctions are tumor-specific and can be used as the tumor-specific biomarkers.

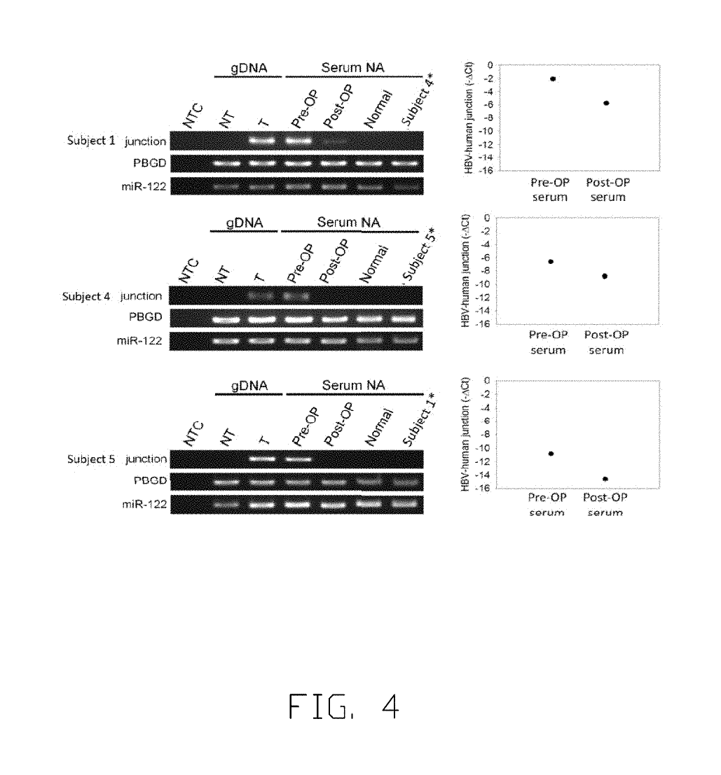

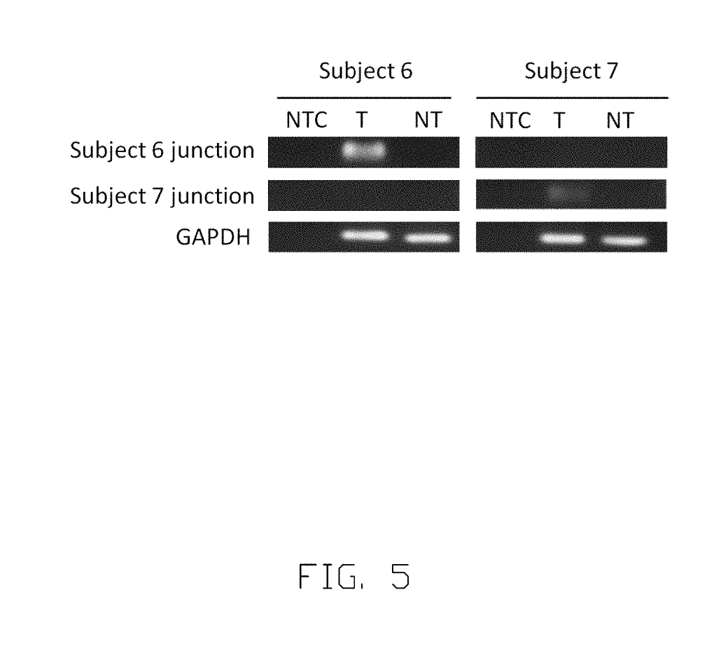

FIG. 4 shows the relationships between tumor size and the amount of specific viral-host junction sequence. Junction sequences used in FIG. 4 for each subject are the same as in FIG. 3. Serial blood samples of each subject are obtained at least at pre-operation and post-operation stages. Referring to FIG. 4, gDNA refers to genomic DNA, NTC refers to no-template control, NT refers to gDNA from non-tumor tissue, T refers to gDNA from tumor tissue, Serum NA refers to DNA obtained from serum, Pre-OP refers to serum DNA obtained at pre-operation stage, Post-OP refers to serum DNA obtained at post-operation stage, Subject 1* refers to serum DNA obtained from Subject 1 and is used in Subject 5 experiment, Subject 4* refers to serum DNA obtained from Subject 4 and is used in Subject 1 experiment, Subject 5* refers to serum DNA obtained from Subject 5 and is used in Subject 4 experiment and Normal refers to serum DNA obtained from a non-patient subject. Serum samples of Subject 1 are obtained at two time points, 13 days before tumor resection (operation) and 19 days after operation. Serum samples of Subject 4 are obtained 33 days before operation and 30 days after operation. Serum samples of Subject 5 are obtained 24 days before operation and 26 days after operation. Serum samples of non-patient subject (Normal) are also included as a control in FIG. 4. As shown in FIG. 4 left panel, the specific viral-host junction of each subject is only present in tumor gDNA and Pre-OP. In addition, the specific viral-host junction of Subject 1 is only present in Subject 1 DNA samples but not in Subject 4 DNA samples, suggesting that the viral-host junction identified is subject-specific. Referring now to the right panel of FIG. 4, the specific viral-host junction in the serum of Subject 1 is detected with relatively large amount in Pre-OP serum while the amount deceases sharply in Post-OP serum. The amount of the specific viral-host junction in Pre-OP serum and Post-OP serum is determined by qPCR and presented in the right panel of FIG. 4. In Subject 1, the amount of specific viral-host junction in Post-OP serum decreases by about 32-fold compared to in Pre-OP serum. Same patterns are observed in Subject 4 and Subject 5, showing that the viral-host junctions or the amount of junctions are tumor-specific, subject-specific, detectable in serum, reflective of the presence and absence of tumor after an operation.

FIG. 5 shows the specificity of viral-host junction sequence. Subject 6 and Subject 7 are processed and analyzed by polymerase chain reaction (PCR). The genomic DNA (gDNA) from tumor tissues and non-tumor tissues is obtained. One chimera DNA sequence in tumor gDNA is identified and selected in each subject to serve as a target to conduct the tests. Specifically the chimera DNA sequence of Subject 6 used in this analysis is AAACGGAAGCATTCTCAGAAACTTCTTGGTGATGTTTGCATTCAAATCCCAGA GTTGAACCTTCCTTTGATAGTTCAGGTTTGAAACACTCTTTCTGTAGGAGACCG CGTAAAGAGAGGTGCGCCCCGTGGTCGGCCGGAACGGCAGATGAAGAAGGG GACGGTAGAGCCCCAAACGGCCCCGAGACG, and the chimera DNA sequence of Subject 7 used in this analysis is TCCCCGCCTAGATCTTTCAGTGAGTCTCTGCCTCAGCTACTCTTAGGATCAGGG GGAGAACCATGGTGTCAGACATCCGGAAAGAAGACGGGATGAATCCAAAACA GGCTTTTACTTTCTCGCCAACTTACAAGGCCTTTCTCAGTGAACAGTATCTGAA CCTTTACCCCGTTGCTCGGCAACGGCCT. The presence of the two specific viral-host junctions can be seen in the tumor gDNA (T) and non-tumor gDNA (NT). NTC refers to no-template control. Glyceraldehyde 3-phosphate dehydrogenase (GAPDH) is used as internal control. As illustrated in FIG. 5, the specific viral-host junction of Subject 6 is only present in tumor gDNA (T) but not in non-tumor gDNA (NT). The specific viral-host junction of Subject 6 is also only present in the samples derived from Subject 6. The specific viral-host junction of Subject 7 is only present in tumor gDNA (T) of Subject 7, but not in non-tumor gDNA (NT) samples of Subject 7 or samples derived from Subject6. Same patterns are observed in Subject 6 and Subject 7, indicating that the identified viral-host junctions are tumor specific and can be used as the tumor-specific biomarkers.

After accumulating read number of junction sequences in patient's serum or plasma in Table 2, 4 and 6, the junction sequence in each of the subjects with the most read number are selected to analyze the concentration of specific viral-host junction sequence in patient's serum or plasma. The specific viral-host junction sequence, and its' read number are identified in Subject 8, 9 and 10 by similar methods conducted in Table 2, 4 and 6. To determine the concentration of the specific viral-host sequence in Subject 8, 9 and 10, the plasma samples are analyzed with BIO-RAD Eva Green droplet digital PCR kit. Each samples diluted and the diluted samples are mixed with a reaction mix containing one or more fluorescence dyes and other reagents, the sample-reaction mix are then subjected QX2000™ Droplet Generator to generation a plurality of small droplets. The droplets are then transferred to C1000 Touch™ Thermal Cycler to conduct polymerase chain reaction. Finally, the QX2000™ Droplet Reader is used to quantify fluorescence signals presented. The fluorescence signal indicated by QX2000™ Droplet Reader provides absolute quantifications of the specific viral-host junctions.

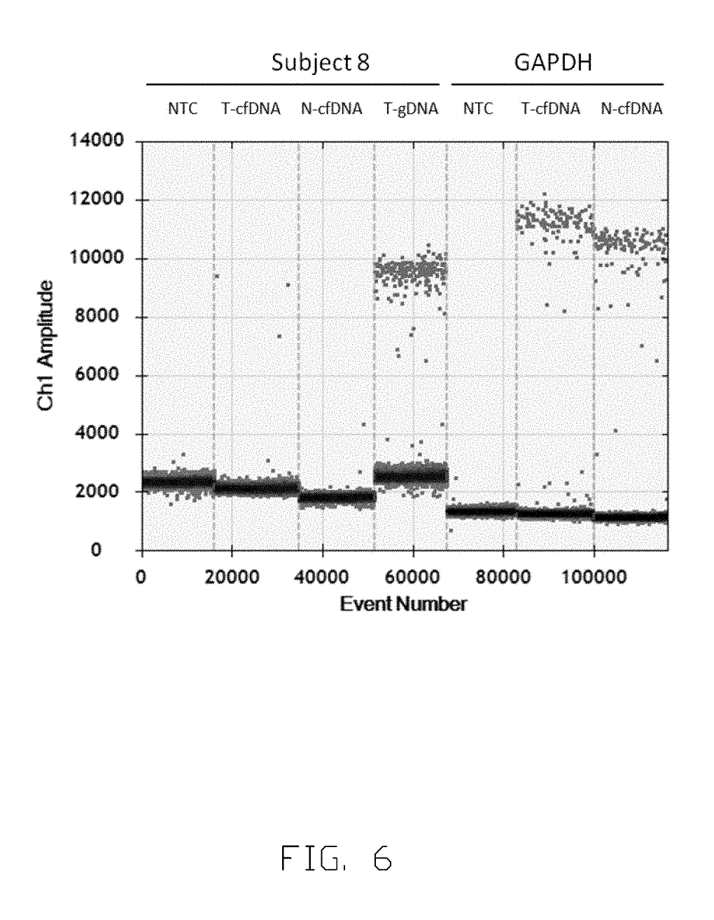

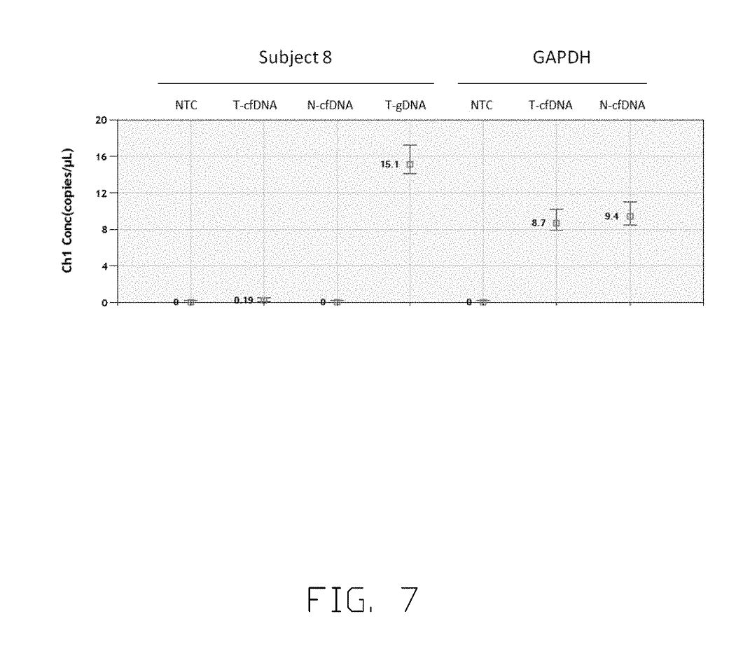

FIG. 6 and FIG. 7 shows the concentration of specific viral-host junction sequence in Subject 8. Droplet digital PCR (ddPCR) analysis is conducted to quantify the concentration of specific viral-host junction sequence in the plasma of Subject 8. Junction sequence quantified in FIGS. 6 and 7 is CTTCAAAGACTGTGTGTTTAATGAGTGGGAGGAGTTGGGGGAGGAGATTAGG TTAAAGGTCTTTGTACTAGGAGGCTGTAGGCATAAATTAAGCGAGAGCCAGGT TGTGGGAAAGCAGGGAAGTGAGGTAGAAGCCTGGTGGCTTTGTGGCTCCATC CCCTCCTCCCTGCCTGCTGCAATAGATACATC. NTC refers to no-template control, T-cfDNA refers to ctDNA in the plasma of Subject 8, N-cfDNA refers to cfDNA in the plasma of a healthy individual and T-gDNA refers to genome DNA from tumor tissue of Subject 8 in FIG. 6 and FIG. 7. Glyceraldehyde 3-phosphate dehydrogenase (GAPDH) is used as internal control in FIG. 6 and FIG. 7. FIG. 6 shows the signals of ctDNA in different sample panels, wherein FIG. 7 shows a consolidated and noise-excluded signals of ctDNA in different sample panels of droplet digital PCR (ddPCR). Referring to FIG. 7, the copy number of ctDNA in T-cfDNA sample is 0.19 copies per one microliter of sample-reaction mix, and there are no ctDNA in NTC and N-cfDNA sample panels. The copy number of ctDNA in T-cfDNA sample are multiplied to calculate the concentration of ctDNA presented in Subject 8. Therefore, the copy number of specific viral-host junction sequence in the plasma of Subject 8 should be 15.2 copies per one milliliter of plasma.

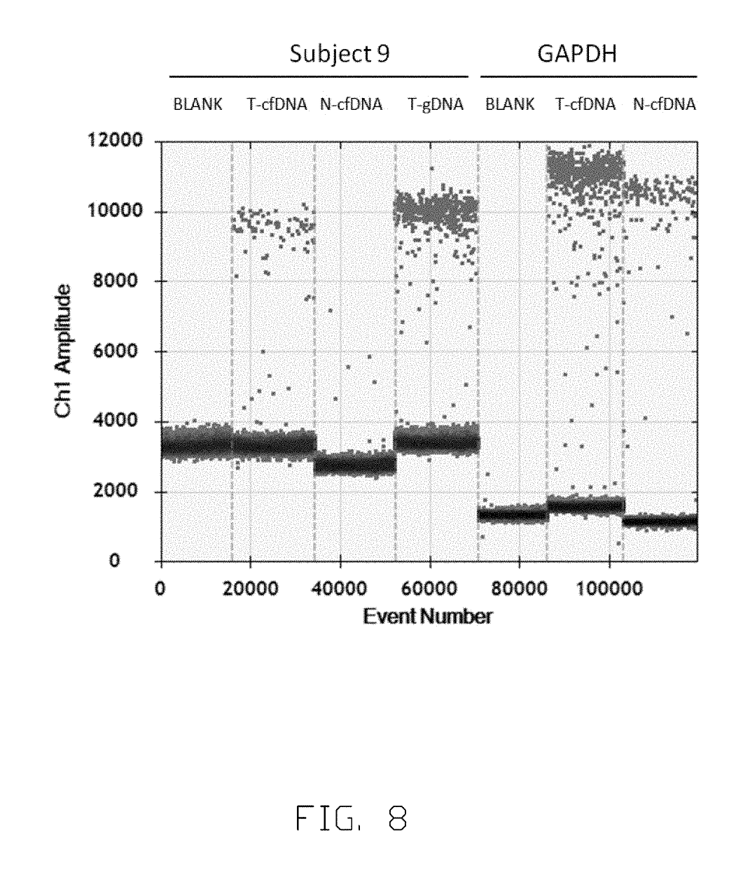

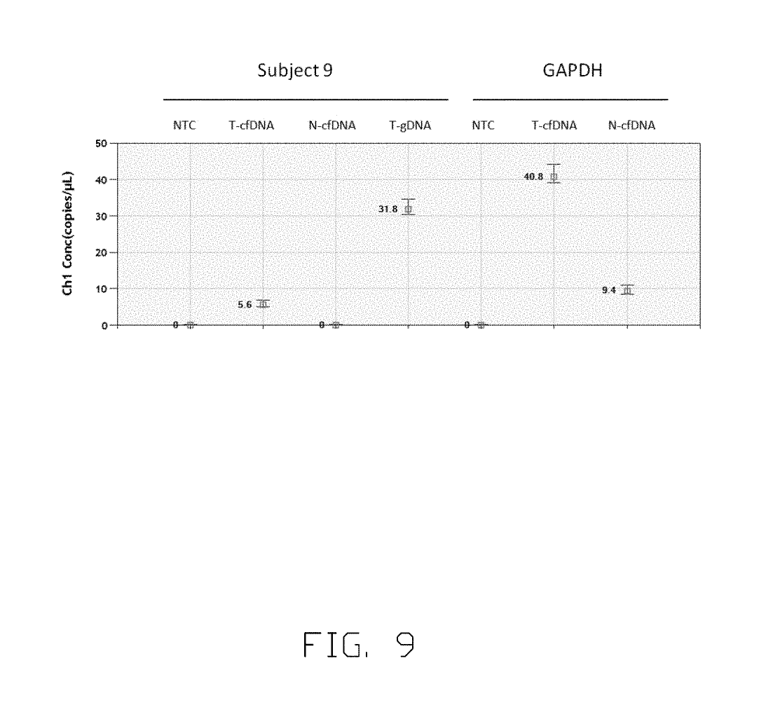

FIG. 8 and FIG. 9 shows the concentration of specific viral-host junction sequence in Subject 9. Droplet digital PCR (ddPCR) analysis is conducted to quantify the concentration of specific viral-host junction sequence in the plasma of Subject 9. Junction sequence quantified in FIG. 8 and FIG. 9 is TTGAGGCATACTTCAAAGACTGTGTGTTTACTGAGTGGGAGGAGTTGGGGGA GGAGATTAGGTTAAAGGTCTTTGTACTAGGAGGCTGTACACTTGCCTCTTCTTT TGCTGACTTCCATGTTCCTCATCGGCCTAGGGTTTCCTGGGGCTGGCTCAACGT CTTACACACTAAATGTTTCACAGTTCACA. NTC refers to no-template control, T-cfDNA refers to ctDNA in the plasma of Subject 9, N-cfDNA refers to non-tumor cfDNA in the plasma of the healthy individual and T-gDNA refers to genome DNA from tumor tissue of Subject9 in FIG. 8 and FIG. 9. Glyceraldehyde 3-phosphate dehydrogenase (GAPDH) is used as internal control in FIG. 8 and FIG. 9. FIG. 8 shows the signals of ctDNA in different sample panels, wherein FIG. 9 shows a consolidated and noise-excluded signals of ctDNA in different sample panels of droplet digital PCR (ddPCR). Referring to FIG. 9, the copy number of ctDNA in T-cfDNA sample is 5.6 copies per one microliter of sample-reaction mix, and there are no ctDNA in NTC and N-cfDNA sample panels. The copy number of ctDNA in T-cfDNA sample are multiplied to calculate the concentration of ctDNA presented in Subject 9. Therefore, the copy number of specific viral-host junction in the plasma of Subject 9 is 448 copies per one milliliter of plasma.

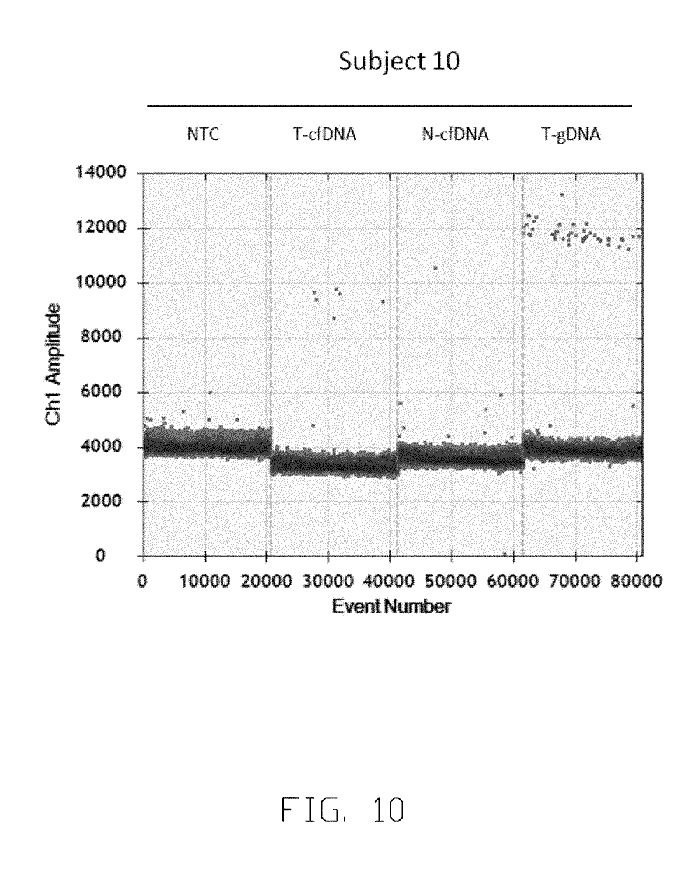

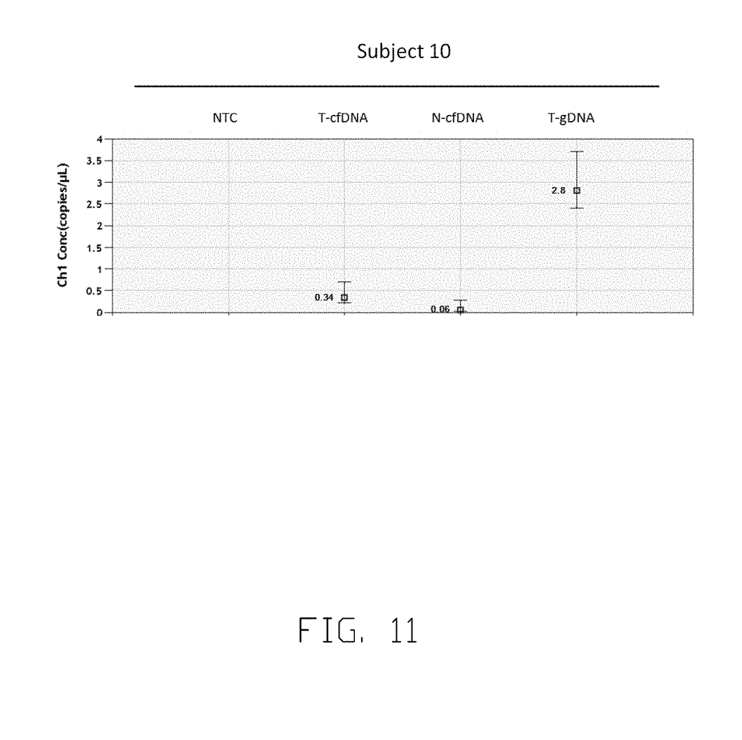

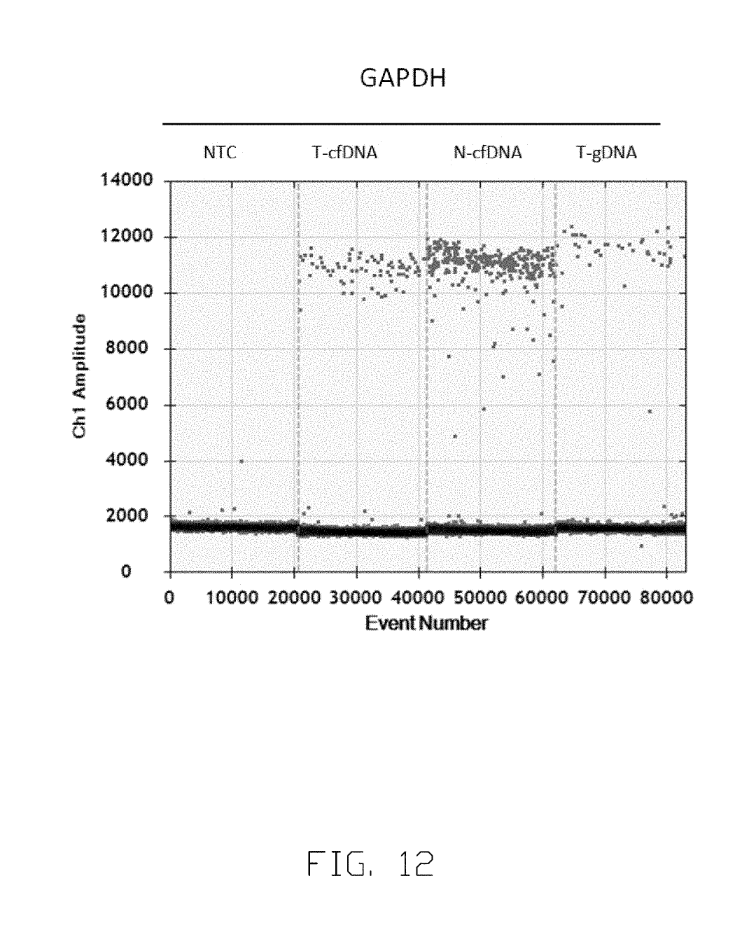

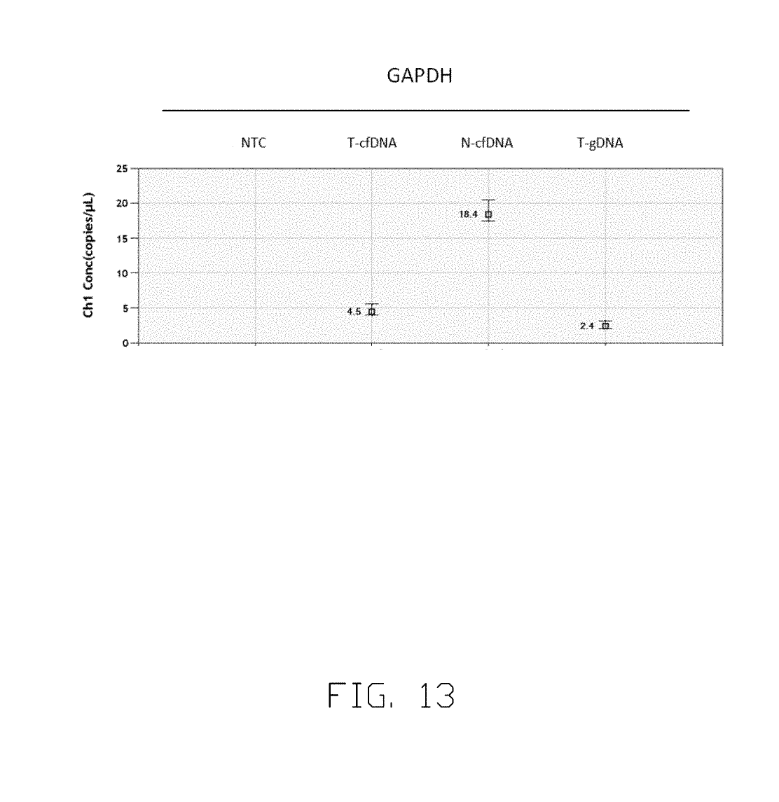

FIG. 10 and FIG. 11 show the concentration of specific viral-host junction sequence in Subject 10. Junction sequence quantified in FIG. 10 and FIG. 11 is GTTGGCTCCGAACGCAGGGTCCAACTGGTGATCGGGAAAGAATCCCAGAGGA TTGGGAACAGAAAGATTCGTCCCCATGCCTTGTCGGGGTTTGGCCCCCAAAGT GCTAGGATAACCCAATCTTTAAACGGGTTAAAGACTTTAATAGACATTTCTCGG CCGGGGGCGGTGGCTCATGCCTGTAATCCTA. Droplet digital PCR (ddPCR) analysis is conducted to quantify the copy number of specific viral-host junction sequence in the plasma. NTC refers to no-template control, T-cfDNA refers to ctDNA in the plasma of Subject 10, N-cfDNA refers to cfDNA in the plasma of the healthy individual and T-gDNA refers to genome DNA from tumor tissue of Subject 10 in FIG. 10, FIG. 11, FIG. 12 and FIG. 13. To serve as an internal control, Glyceraldehyde 3-phosphate dehydrogenase (GAPDH) are used in FIG. 12 and FIG. 13, and GAPDH are presented in T-cfDNA, N-cfDNA and T-gDNA. FIG. 10 shows the signals of ctDNA in different sample panels, wherein FIG. 11 shows a consolidated and noise-excluded signals of ctDNA in different sample panels of droplet digital PCR (ddPCR). Referring to FIG. 11, the copy number of ctDNA in T-cfDNA sample is 0.34 copies per one microliter of sample-reaction mix, and there are no ctDNA in NTC sample panels. The copy number of ctDNA in T-cfDNA sample are multiplied to calculate the concentration of ctDNA presented in Subject 10. Therefore, the copy number of specific viral-host junction in the plasma of Subject 10 is 29 copies per one milliliter of plasma.

Table 7 shows the information of Subject 8, 9 and 10, including the gender, age, tumor size and ctDNA junction concentration.

| | | | Tumor | |
|---|---|---|---|---|
| | | | size | ctDNA junction concentration |
| Sample | Gender | Age | (cm³) | (copy number per ml) |
| Subject 8 | M | 52 | 3 | 15.2 |
| Subject 9 | M | 66 | 11.58 | 448 |
| Subject 10 | M | 43 | 4.6 | 29 |

The above results from Subject 8, Subject 9 and Subject 10 suggest a relationship of tumor size. The copy number of specific viral-host junctions can be inferred. The tumor size is positively correlated to the concentration of ctDNA junctions. The larger tumor size represents higher copy number of ctDNA junction in patient's blood. The presence of ctDNA junction in patient's serum or plasma can be indicative of tumor status. Specifically, the copy number of ctDNA junction in patient's blood can be used to monitor the size of tumor within one patient when evaluating the prognosis after the surgical removal, radiotherapy, chemotherapy or other therapeutic approach on the tumor. The copy number of ctDNA junction in patient's blood can also be used to assess the size of tumor when diagnosing the tumor status. The ctDNA junction concentration in the plasma or serum of more than 30 copies per milliliter of plasma or serum may represent a tumor size of more than 4.6 cm³. The relationship between the ctDNA junction concentration in the plasma or serum and the tumor size may be an exponential function or a linear function.

The read number of ctDNA junction in patient's serum or plasma provides a non-invasive diagnosis for tumor. Therefore, the read number of ctDNA junction can be used to diagnose the presence of tumor to a patient with hepatitis-B virus infection, or to evaluate the tumor status before surgical removal, radiotherapy, chemotherapy of other therapeutic approach on the tumor. The read number of ctDNA junction in patient's blood can also be used to assess the presence and the size of tumor.

It is to be noted that by using the approach described in the present invention, mutated p53 or beta-catenin genes cannot be detected in the ctDNAs despite the mutations are identified in the tumor tissues (data not shown). The result shows that by using the method of present invention, tumor specific viral-host junctions (viral genome sequence insertion into host genome), and not conventional somatic mutations, are selectively enriched and obtained to provide cancer/tumor information.

The embodiments shown and described above are only examples. Many details are often found in the art for example the other features of a circuit board assembly. Therefore, many such details are neither shown nor described. Even though numerous characteristics and advantages of the present technology have been set forth in the foregoing description, together with details of the structure and function of the present disclosure, the disclosure is illustrative only and changes may be made in the detail, including in matters of shape, size and arrangement of the parts within the principles of the present disclosure up to, and including the full extent established by the broad general meaning of the terms used in the claims. It will therefore be appreciated that the embodiments described above may be modified within the scope of the claims.

What is claimed is:

1. A method of identifying an amount of viral-host junction sequences from a subject with a hepatitis B virus infection, comprising:
obtaining a cfDNA in a serum or plasma from the subject with a hepatitis B virus infection;
ligating the cfDNA with at least one adaptor;
amplifying the cfDNA ligated with the at least one adaptor by using a plurality of primers, wherein each of the primers is complementary to a sequence of the corresponding adaptor;
hybridizing at least one polynucleotide probe with the cfDNA ligated with the at least one adaptor;
capturing and isolating a target ctDNA in the cfDNA hybridized with the at least one polynucleotide probe;
sequencing the target ctDNA by a sequencing system, wherein the target ctDNA has one or more of the viral-host junction sequences, wherein each of the viral-host junction sequences comprises a hepatitis B viral genome sequence and a host genome sequence; and
accumulating a read number related to the viral-host junction sequences in the target ctDNA to identify the viral-host junction sequences by the sequencing system, wherein an amount of one of the viral-host junction sequences is defined as a read number, the read number is defined as times of the viral-host junction sequences appearing in the target ctDNA, wherein the detected viral-host junction sequences comprise same sequences, different sequences, or a combination thereof, and each of the viral-host junction sequences is counted to give the read number, and wherein the amount of one of the viral-host junction sequences is configured to indicate a presence of a hepatitis B virus-related tumor in the subject with the hepatitis B virus infection.

2. The method of claim 1, wherein the target ctDNA is enriched by the at least one polynucleotide probe complementary to at least one part of the sequence derived from hepatitis B viral genome.

3. The method of claim 1, wherein at least two of the polynucleotide probes cover the whole hepatitis B viral genome sequence.

4. The method of claim 1, wherein the cfDNA ligating with the corresponding adaptor comprises at least one of a first ctDNA with one end thereof ligating with the corresponding adaptor and a second cfDNA with two ends thereof ligating with the corresponding adaptors.

5. The method of claim 1, wherein the hepatitis B virus-related tumor is a hepatocellular carcinoma induced by the hepatitis B virus.

6. A method of monitoring tumor size from a serum or plasma sample derived from a subject with a hepatitis B virus-related tumor, comprising:
obtaining a cfDNA in a serum or plasma from the subject with the hepatitis B virus-related tumor;
ligating the cfDNA with at least one adaptor;
amplifying the cfDNA ligated with the at least one adaptor by using a plurality of primers, wherein each of the primers is complementary to a sequence of the corresponding adaptor;
hybridizing at least one polynucleotide probe with the cfDNA ligated with the adaptor;
capturing and isolating a target ctDNA in the cfDNA hybridized with the at least one polynucleotide probe;
sequencing the target ctDNA by a sequencing system, wherein the target ctDNA has one or more viral-host junction sequences, wherein each of the viral-host junction sequences comprises a hepatitis B viral genome sequence and a host genome sequence; and
accumulating a read number related to the viral-host junction sequences in the target ctDNA to identify the viral-host junction sequences by the sequencing system, wherein an amount of one of the viral-host junction sequences is defined as a read number, the read number is defined as times of the viral-host junction sequences appearing in the target ctDNA, wherein the detected viral-host junction sequences comprise same sequences, different sequences, or a combination thereof, and each of the viral-host junction sequences is counted to give the read number; and
quantifying a specific viral-host junction sequence by a quantification system, wherein the specific viral-host junction sequence is one of the viral-host junction sequence with a highest read number, and wherein a concentration of the specific viral-host junction sequence is configured to indicate a size of the hepatitis B virus-related tumor.

7. The method of claim 6, wherein the target ctDNA is enriched by the at least one polynucleotide probe complementary to at least one part of the sequence derived from hepatitis B viral genome.

8. The method of claim 6, wherein at least two of the polynucleotide probes cover the whole hepatitis B viral genome sequence.

9. The method of claim 6, wherein the cfDNA ligating with the corresponding adaptor comprises at least one of a first ctDNA with one end thereof ligating with the corresponding adaptor and a second cfDNA with two ends thereof ligating with the corresponding adaptors.

10. The method of claim 6, wherein the hepatitis B virus-related tumor is a hepatocellular carcinoma induced by a hepatitis B virus.

11. The method of claim 6, wherein the concentration of the one or more viral-host junction sequences in the target ctDNA of the subject is positively correlated to the size of the tumor in the subject.


SEQUENCE LISTING (63 sequences)

SEQ ID NO: 1 - DNA, 40 bp, Homo sapiens
ggtcttacataagaggactcagaaaatactttgtgatgat

SEQ ID NO: 2 - DNA, 40 bp, Homo sapiens
aactccttttgagagcgcagtgttcggtgcaggtccccag

SEQ ID NO: 3 - DNA, 40 bp, Homo sapiens
atcatcacaaagtattttctgagtcctcttatgtaagacc

SEQ ID NO: 4 - DNA, 40 bp, Homo sapiens
tgaggtgagaggatctcttgagcacagatgatgggatagg

SEQ ID NO: 5 - DNA, 40 bp, Homo sapiens
aaacgtccacttgcagattttatgtaattggaagttgggg

SEQ ID NO: 6 - DNA, 40 bp, Homo sapiens
agcaggaaaatatatgccccaccttccctttctctgaccc

SEQ ID NO: 7 - DNA, 40 bp, Homo sapiens
aggaagactgcctactcccacaggcctgaaagcgctccaa

SEQ ID NO: 8 - DNA, 40 bp, Homo sapiens
agcattcgggccagggttcactcaggctcagggcacattg

SEQ ID NO: 9 - DNA, 40 bp, Homo sapiens
gcatttggtggtctataagcacacccgcccacaccaatct

SEQ ID NO: 10 - DNA, 40 bp, Homo sapiens
caagaccagcctgaggatgactgtctcttagaggtggaga

SEQ ID NO: 11 - DNA, 40 bp, Homo sapiens
cactccttttgagagcgcagtgttcaggtgcagggtcccc

SEQ ID NO: 12 - DNA, 40 bp, Homo sapiens; identical to SEQ ID NO: 3

SEQ ID NO: 13 - DNA, 40 bp, Homo sapiens
aacagaaagattcgtccccaaatccaatctgtcttccatc

SEQ ID NO: 14 - DNA, 40 bp, Homo sapiens
agcaggaaaatatatgccccaccttccctttctctgccct

SEQ ID NO: 15 - DNA, 40 bp, Homo sapiens; identical to SEQ ID NO: 1

SEQ ID NO: 16 - DNA, 40 bp, Homo sapiens; identical to SEQ ID NO: 9

SEQ ID NO: 17 - DNA, 40 bp, Homo sapiens
atcatcctgggctttctgcacttcccataggtaatcaaag

SEQ ID NO: 18 - DNA, 40 bp, Homo sapiens; identical to SEQ ID NO: 8

SEQ ID NO: 19 - DNA, 100 bp, Homo sapiens
atgaagctatttataataaaacaaactttattaaatctagtttaaatgccttactctcttttttgccttctgacttctttccttctattcgagatctcct

SEQ ID NO: 20 - DNA, 100 bp, Homo sapiens
tttcattgttgctgtttttcaaattgattttgggatccagcctgttattctactcccttaacttcatgggatatgtaattggaagttggggtactttacc

SEQ ID NO: 21 - DNA, 100 bp, Homo sapiens
tctccctttagacttcaaacacttcaaaatatgacttcactacaaagctttatagaatgccagccttccacagagtatgtaaataatgcctagttttgaa

SEQ ID NO: 22 - DNA, 100 bp, Homo sapiens
ccagcacatttgtctataaatttacattcttggatattagcaaaattgcaaacagaccaatttatgcctacagcctcctagtacaaagacctttaaccta

SEQ ID NO: 23 - DNA, 100 bp, Homo sapiens
tccagtgtttgtgggttgagcagtattattgcatggcccagtggtggtggttgatgttcctggaagtagaggacaaacgggcaacataccttggtagtcc

SEQ ID NO: 24 - DNA, 100 bp, Homo sapiens
tgcaagtggttgcagttcttttgctttgccaccaccactgggccatgcaaaacctgcacgattcctgctcaaggaacctctatgtttccctcttgttgct

SEQ ID NO: 25 - DNA, 100 bp, Homo sapiens
caggaggaggtgatggacccactgggtggtgaagaacagtttctcttccaaaattacttcccacccaggtggccagattcatcaactcaccccaacacag

SEQ ID NO: 26 - DNA, 100 bp, Homo sapiens
atctgtaaaattgggatcatcacactttccttttattggggtttaaatgaatacccaaagacaaaagaaaattggtaatagaggtaaaaagggactcaag

SEQ ID NO: 27 - DNA, 100 bp, Homo sapiens
tggccgaggccatcttctaaataaatgtgtggaagagaaactgttcttcagtatttggtgtcttttggagtgtggattcgcactcctcccgcttacagac

SEQ ID NO: 28 - DNA, 100 bp, Homo sapiens
aggacgggtgcccgggtccccagtccctccgccacgtgggaagcgcggtccagaccaatttatgcctacagcctcctagtacaaagacctttaacctaat

SEQ ID NO: 29 - DNA, 100 bp, Homo sapiens; identical to SEQ ID NO: 19

SEQ ID NO: 30 - DNA, 100 bp, Homo sapiens; identical to SEQ ID NO: 25

SEQ ID NO: 31 - DNA, 100 bp, Homo sapiens; identical to SEQ ID NO: 23

SEQ ID NO: 32 - DNA, 100 bp, Homo sapiens
gtccgttggtggtgaactgggcaagataattgcatggcccagtggtggtggttgatgttcctggaagtagaggacaaacgggcaacataccttggtagtc

SEQ ID NO: 33 - DNA, 100 bp, Homo sapiens
agattggtctataattttcttttactatcttcagtatttggtatctttgggagtgtggattcgcactcctcccgcttacagaccaccaaatgcccctatc

SEQ ID NO: 34 - DNA, 100 bp, Homo sapiens; identical to SEQ ID NO: 20

SEQ ID NO: 35 - DNA, 100 bp, Homo sapiens; identical to SEQ ID NO: 27

SEQ ID NO: 36 - DNA, 100 bp, Homo sapiens; identical to SEQ ID NO: 24

SEQ ID NO: 37 - DNA, 100 bp, Homo sapiens
gtaagccattgtggctttcctgaccagcccaccaccactgggccatgcaaaacctgcacgattcctgctcaaggaacctctatgtttccctcttgttgct

SEQ ID NO: 38 - DNA, 100 bp, Homo sapiens; identical to SEQ ID NO: 22

SEQ ID NO: 39 - DNA, 100 bp, Homo sapiens
ggaaatggagccaggcgctcctgctggccgcgcaccgggcgcctcacaccagaacatcgcatcaggactcctaggacccctgctcgtgttacaggcgggg

SEQ ID NO: 40 - DNA, 100 bp, Homo sapiens
tcaagcagaaaaaccatgaagatttaaaaacttgtaaatatttgaatgtgggctccaccccaacagtcccccgtggggaggggtgaaccctggcccgaat

SEQ ID NO: 41 - DNA, 100 bp, Homo sapiens
ctaagggacactacaggaaaccagccccgaagtgatttcttttgaaattccaaatctttctgtccccaatcccctgggattcttccccgatcatcagttg

SEQ ID NO: 42 - DNA, 100 bp, Homo sapiens
cctcgaagcctgtgccaacctagcccattcctcaggctcagggcctcctcacatctgtgccagcagctcctcctcctgcctccaccaatcggcagtcagg

SEQ ID NO: 43 - DNA, 100 bp, Homo sapiens
cattgttactgtgatatgctataattattctcaccttatgtgtccaaggaatactaacattgagattcccgagattgagatcttctgcgacgcggcgatt

SEQ ID NO: 44 - DNA, 100 bp, Homo sapiens
atggagaatacagcacattattaggagtaagtttccttaaacacattttgattttttgtacaatatgttcctgtggcaatgtgccccaactcccaattac

SEQ ID NO: 45 - DNA, 100 bp, Homo sapiens
tttgccaccttcctgccactttgtagatgcaagatcttgggcaagttcccgtgggcgttcacggtggtttccatgcgacgtgcagaggtgaagcgaagtg

SEQ ID NO: 46 - DNA, 100 bp, Homo sapiens
cagtggaaacaaagccactgggaagttcaaactgagagaagcccaccacaagtctagactctgtggtattgtgaggatttttgtcaacaagaaaaacccc

SEQ ID NO: 47 - DNA, 100 bp, Homo sapiens
agtatatcatcagttatttttcaaggttttctaagtaaacagtttctcaacctttaccccgttgctcggcaacggcctggtctgtgccaagtgtttgctg

SEQ ID NO: 48 - DNA, 100 bp, Homo sapiens
tcagggaggggatgttgactgcattttggaggttcagggcctactaacaactgtgccagcagctcctcctcctgcctccaccaatcggcagtcaggaagg

SEQ ID NO: 49 - DNA, 100 bp, Homo sapiens; identical to SEQ ID NO: 39

SEQ ID NO: 50 - DNA, 100 bp, Homo sapiens; identical to SEQ ID NO: 40

SEQ ID NO: 51 - DNA, 100 bp, Homo sapiens
cccgggaccgaccccaggaagagccaggggcccgggtgatccctgcgggggtctggctttcagttatatggatgatgtggtattgggggccaagtctgta

SEQ ID NO: 52 - DNA, 100 bp, Homo sapiens
aatgaaaatctcattgatttttcacttataggttttaccttagagctcctcctctgcctaatcatctcatgttcatgtcctactgttcaagcctccaagc

SEQ ID NO: 53 - DNA, 100 bp, Homo sapiens
agaattgatacctaagctgagcagaaatgaggccgaccatgaagtgagtgcctaatcatctcatgttcatgtcctactgttcaagcctccaagctgtgcc

SEQ ID NO: 54 - DNA, 100 bp, Homo sapiens
cgtaggaaagacaaggtggcattgatggaaagcagtagtttttgagcccttcgcagacgaaggtctcaatcgccgcgtcgcagaagatctcaatctcggg

SEQ ID NO: 55 - DNA, 100 bp, Homo sapiens
ttaaaaaggagttttgtttgttagtctattcactcatttcaaggaacatagaagaagaactccctcgcctcgcagacgaaggtctcaatcgccgcgtcgc

SEQ ID NO: 56 - DNA, 100 bp, Homo sapiens
cagttccctggctccaagctccctcaaaagatgcccagctggcctttcccaaaggccttgtaagttggcgagaaagtaaaagcctgttttgcttgtatac

SEQ ID NO: 57 - DNA, 100 bp, Homo sapiens
acatgcccttcacttcagcctgatgctcctggcataagctcagcaattttggagtgcgaatccacactccaaaagacaccaaatattcaagaacagtttc

SEQ ID NO: 58 - DNA, 100 bp, Homo sapiens
aatttcccctgaatagctgcagtactcacagacacactggatgctactcacctctgcctaatcatctcatgttcatgtcctactgttcaagcctccaagc

SEQ ID NO: 59 - DNA, 188 bp, Homo sapiens
aaacggaagcattctcagaaacttcttggtgatgtttgcattcaaatcccagagttgaaccttcctttgatagttcaggtttgaaacactctttctgtaggagaccgcgtaaagagaggtgcgccccgtggtcggccggaacggcagatgaagaaggggacggtagagccccaaacggccccgagacg

SEQ ID NO: 60 - DNA, 188 bp, Homo sapiens
tccccgcctagatctttcagtgagtctctgcctcagctactcttaggatcagggggagaaccatggtgtcagacatccggaaagaagacgggatgaatccaaaacaggcttttactttctcgccaacttacaaggcctttctcagtgaacagtatctgaacctttaccccgttgctcggcaacggcct

SEQ ID NO: 61 - DNA, 189 bp, Homo sapiens
cttcaaagactgtgtgtttaatgagtgggaggagttgggggaggagattaggttaaaggtctttgtactaggaggctgtaggcataaattaagcgagagccaggttgtgggaaagcagggaagtgaggtagaagcctggtggctttgtggctccatcccctcctccctgcctgctgcaatagatacatc

SEQ ID NO: 62 - DNA, 189 bp, Homo sapiens
ttgaggcatacttcaaagactgtgtgtttactgagtgggaggagttgggggaggagattaggttaaaggtctttgtactaggaggctgtacacttgcctcttcttttgctgacttccatgttcctcatcggcctagggtttcctggggctggctcaacgtcttacacactaaatgtttcacagttcaca

SEQ ID NO: 63 - DNA, 190 bp, Homo sapiens
gttggctccgaacgcagggtccaactggtgatcgggaaagaatcccagaggattgggaacagaaagattcgtccccatgccttgtcggggtttggcccccaaagtgctaggataacccaatctttaaacgggttaaagactttaatagacatttctcggccgggggcggtggctcatgcctgtaatccta